(19) United States
(12) Patent Application Publication
(10) Pub. No.: US 2001/0032649 A1
(43) Pub. Date: Oct. 25, 2001

(54) Intruded object and magnetic resonance imaging apparatus

(76) Inventor: Shigeo Nagano, Tokyo (JP)

Correspondence Address: MOONRAY KOJIMA, BOX 627, WILLIAMSTOWN, MA, 01267, US

(21) Appl. No.: 09/790,154

(22) Filed: Feb. 21, 2001

(30) Foreign Application Priority Data
Feb. 29, 2000 (JP) 2000-52677

(51) Int. Cl. A61B 19/00

(52) U.S. Cl. 128/897

(57) ABSTRACT

With an object of realizing an intruded object capable of carrying out magnetic resonance imaging in a state of being intruded into an object and a magnetic resonance imaging apparatus for capturing an image of the object having the intruded object, at least a unit of an intruded object 400 such as a piercing needle intruded into an inner unit of the object, is constituted by a substance 402 or 404 having an atomic nucleus having a gyromagnetic ratio different from a gyromagnetic ratio of spin of an atomic nucleus of a substance occupying a majority in the object.

### BACKGROUND OF THE INVENTION

The present invention relates to an intruded object and a magnetic resonance imaging apparatus, particularly to a body intruded to an object for carrying out magnetic resonance imaging and a magnetic resonance imaging apparatus for capturing an image of the object intruded with the body.

According to MRI (Magnetic Resonance Imaging) apparatus, an object an image of which is to be taken is carried into an inner space of a magnet system, that is, a space formed with a magnetostatic field, a magnet resonance signal is generated in the object by applying a gradient magnetic field as well as a high-frequency magnetic field and a laminar image is formed (reconstructed) based on a received signal thereof.

When there is carried out intervention such as biopsy of tissue, an object is pierced while observing a laminar image taken in real time. In that case, a piercing needle cannot be visualized and therefore, piercing is carried out by depending on an angular graduation provided at a piercing needle guide or a length graduation provided at the piercing needle.

According to the above-described piercing operation, a state of the piercing needle in the body cannot optically be recognized and accordingly, the operational performance is poor.

### SUMMARY OF THE INVENTION

It is an object of the present invention to provide an intruded object capable of carrying out magnetic resonance imaging under a state of being intruded to an object and a magnetic resonance imaging apparatus for capturing an image of an object having such an intruded object.

(1) According to an aspect of the present invention to resolve the above-described problem, there is provided an intruded object which is a body intruded into an inner unit of an object for executing magnetic resonance imaging, characterized in that at least a unit of the body comprises a substance having an atomic nucleus having a gyromagnetic ratio different from a gyromagnetic ratio of spin of an atomic nucleus of a substance occupying a majority in the object.

According to the aspect of the invention, at least a unit of the intruded object is constituted by the substance having the atomic nucleus having the gyromagnetic ratio different from the gyromagnetic ratio of spin of the atomic nucleus of the substance occupying the majority in the object and accordingly, the object and the intruded object can be differentiated from each other by frequencies of magnetic resonance signals.

(2) According to another aspect of the present invention to resolve the above-described problem, there is provided the intruded object according to (1), characterized in that the substance is harmless to an organism.

According to the aspect of the present invention, the substance harmless to an organism is used and accordingly, the safety can be promoted.

(3) According to another aspect of the present invention to resolve the above-described problem, there is provided the intruded object according to (2), characterized in that the substance is silicon.

According to the aspect of the present invention, at least a unit of the intruded object is constituted by silicon and accordingly, the object and the intruded object can be differentiated from each other by frequencies of the magnetic resonance signals.

(4) According to another aspect of the present invention to resolve the above-described problem, there is provided the intruded object according to (2), characterized in that the substance is N-acetylasparatate.

According to the aspect of the present invention, at least a unit of the intruded object is constituted by N-acetylasparatate and accordingly, the object and the intruded object can be differentiated from each other by frequencies of the magnetic resonance signals.

(5) According to another aspect of the present invention to resolve the above-described problem, there is provided the intruded object according to (1), characterized in that the substance comprises a plurality of substances having different gyromagnetic ratios of spin of atomic nuclei.

According to the aspect of the present invention, at least a unit of the intruded object is constituted by the plurality of substances having different gyromagnetic ratios of spin of atomic nuclei and accordingly, by frequencies of the magnetic resonance signals, the intruded object and the object can be differentiated from each other and the plurality of substances can be differentiated from each other.

(6) According to another aspect of the present invention to resolve the above-described problem, there is provided the intruded object according to (5), characterized in that all of the plurality of substances are harmless to an organism.

According to the aspect of the present invention, the substance harmless to an organism is used and accordingly, the safety can be promoted.

(7) According to another aspect of the present invention to resolve the above-described problem, there is provided the intruded object according to (6), characterized in that one of the plurality of substances is silicon and other thereof is N-acetylasparatate.

According to the aspect of the present invention, at least units of the intruded object are constituted by silicon and N-acetylasparatate and accordingly, by frequencies of the magnetic resonance signals, the intruded object and the object can be differentiated from each other and the plurality of substances can be differentiated from each other.

(8) According to another aspect of the present invention to resolve the above-described problem, there is provided the intruded object according to any one of (1) through (7), characterized in that the body pierces the object.

According to the aspect of the present invention, at least a unit of a pierced object is constituted by a substance having an atomic nucleus having a gyromagnetic ratio different from a gyromagnetic ratio of spin of an atomic nucleus of a substance occupying a majority in the object and accordingly, the object and the pierced object can be differentiated from each other by frequencies of the magnetic resonance signals.

(9) According to another aspect of the present invention to resolve the above-described problem, there is provided the intruded object according to any one of (1) through (7), characterized in that the body is inserted into a celom of the object.

According to the aspect of the present invention, at least a unit of an object inserted into the celom is constituted by a substance having an atomic nucleus having a gyromagnetic ratio different from a gyromagnetic ratio of spin of an atomic nucleus of a substance occupying a majority in the object and accordingly, the object and the object inserted into the celom can be differentiated from each other by frequencies of the magnetic resonance signals.

(10) According to another aspect of the present invention to resolve the above-described problem, there is provided the intruded object according to any one of (1) through (7), characterized in that the body is embedded in the object.

According to the aspect of the present invention, at least a unit of an embedded object is constituted by a substance having an atomic nucleus having a gyromagnetic ratio different from a gyromagnetic ratio of spin of an atomic nucleus of a substance occupying a majority in the object and accordingly, the object and the embedded object can be differentiated from each other by frequencies of the magnetic resonance signals.

(11) According to another aspect of the present invention to resolve the above-described problem, there is provided a magnetic resonance imaging apparatus characterized in comprising signal acquiring means for acquiring magnetic resonance signals respectively of an object for executing magnetic resonance imaging and a body which is intruded into an inner unit of the object and at least a unit of which comprises a substance having an atomic nucleus having a gyromagnetic ratio different from a gyromagnetic ratio of spin of an atomic nucleus of a substance occupying a majority in the object, and image forming means for forming respectively an image of the object and an image of the intruded object based on the magnetic resonance signals.

According to the aspect of the present invention, at least a unit of the intruded object is constituted by the substance having the atomic nucleus having the gyromagnetic ratio different from the gyromagnetic ratio of spin of the atomic nucleus of the substance occupying the majority in the object and accordingly, the object and the intruded object can be differentiated from each other by frequencies of the magnetic resonance signals. Thereby, the magnetic resonance imaging can be executed to differentiate the object from the intruded object.

(12) According to another aspect of the present invention to resolve the above-described problem, there is provided the magnetic resonance imaging apparatus according to (11), characterized in that the signal acquiring means alternately acquires the magnetic resonance signals with respect to the object and the body.

According to the aspect of the present invention, the magnetic resonance imaging can be carried out to differentiate the object from the intruded object by alternately acquiring the magnetic resonance signals with respect to the object and the body.

(13) According to another aspect of the present invention to resolve the above-described problem, there is provided the magnetic resonance imaging apparatus according to (11) or (12), characterized in further comprising image displaying means for displaying the image of the object and the image of the intruded object on a same screen to differentiate from each other.

According to the aspect of the present invention, the image of the object and the image of the intruded object are displayed to differentiate from each other on the same screen by the image displaying means and accordingly, an observer can recognize a state of the intruded object at an inner unit of the object.

(14) According to another aspect of the present invention to resolve the above-described problem, there is provided the magnetic resonance imaging apparatus according to any one of (11) through claim (13), characterized in further comprising position displaying means for displaying a position of the intruded object based on the image of the intruded object.

According to the aspect of the present invention, the position of the intruded object is displayed by the position displaying means and accordingly, the observer can recognize the position of the intruded object at the inner unit of the object.

(15) According to another aspect of the present invention to resolve the above-described problem, there is provided the magnetic resonance imaging apparatus according to any one of (11) through (14), characterized in that the substance is harmless to an organism.

According to the aspect of the present invention, the substance harmless to an organism is used and accordingly, the safety can be promoted.

(16) According to another aspect of the present invention to resolve the above-described problem, there is provided the magnetic resonance imaging apparatus according to (15), characterized in that the substance is silicon.

According to the aspect of the present invention, at least a unit of the intruded object is constituted by silicon and accordingly, the object and the intruded object can be differentiated from each other by frequencies of the magnetic resonance signals. Thereby, the magnetic resonance imaging can be executed to differentiate the object from the intruded object.

(17) According to another aspect of the present invention to resolve the above-described problem, there is provided the magnetic resonance imaging apparatus according to (15), characterized in that the substance is N-acetylasparatate.

According to the aspect of the present invention, at least a unit of the intruded object is constituted by N-acetylasparatate and accordingly, the object and the intruded object can be differentiated from each other by frequencies of the magnetic resonance signals. Thereby, the magnetic resonance imaging can be executed to differentiate the object from the intruded object.

(18) According to another aspect of the present invention to resolve the above-described problem, there is provided the magnetic resonance imaging apparatus according to any one of (11) through (14), characterized in that the substance comprises a plurality of substances having different gyromagnetic ratios of spin of atomic nuclei.

According to the aspect of the present invention, at least units of the intruded object are constituted by the plurality of substances having different gyromagnetic ratios of spin of atomic nuclei and accordingly, the intruded object and the object can be differentiated from each other by frequencies of the magnetic resonance signals and the plurality of substances can be differentiated from each other. Thereby, the magnetic resonance imaging can be executed to differentiate the object from the intruded object and differentiate respectively the plurality of substances from each other.

(19) According to another aspect of the present invention to resolve the above-described problem, there is provided the magnetic resonance imaging apparatus according to (18), characterized in that all of the plurality of substances are harmless to an organism.

According to the aspect of the present invention, the substance harmless to an organism is used and accordingly, the safety can be promoted.

(20) According to another aspect of the present invention to resolve the above-described problem, there is provided the magnetic resonance imaging apparatus according to (19), characterized in that one of the plurality of substances is silicon and other thereof is N-acetylasparatate.

According to the aspect of the present invention, at least units of the intruded object are constituted by silicon and N-acetylasparatate and accordingly, by frequencies of the magnetic resonance signals, the intruded object and the object can be differentiated from each other and the plurality of substances can be differentiated from each other respectively. Thereby, the magnetic resonance imaging can be carried out to differentiate the object from the intruded object and differentiate the plurality of substance from each other.

(21) According to another aspect of the present invention to resolve the above-described problem, there is provided the magnetic resonance imaging apparatus according to any one of (11) through (20), characterized in that the body pierces the object.

According to the aspect of the present invention, at least a unit of a pierced object is constituted by a substance having an atomic nucleus having a gyromagnetic ratio different from a gyromagnetic ratio of spin of an atomic nucleus of a substance occupying majority in the object and accordingly, the object and the pierced object can be differentiated from each other by frequencies of the magnetic resonance signals. Thereby, the magnetic resonance imaging can be carried out by differentiating the object from the pierced object.

(22) According to another aspect of the present invention to resolve the above-described problem, there is provided the magnetic resonance imaging apparatus according to any one of (11) through (20), characterized in that the body is inserted into a celom of the object.

According to the aspect of the present invention, at least a unit of an object inserted into the celom is constituted by a substance having an atomic nucleus having a gyromagnetic ratio different from a gyromagnetic ratio of spin of an atomic nucleus of a substance occupying a majority in the object and accordingly, the object and the object inserted into the celom can be differentiated from each other by frequencies of the magnetic resonance signals. Thereby, the magnetic resonance imaging can be executed to differentiate the object from the object inserted into the celom.

(23) According to another aspect of the present invention to resolve the above-described problem, there is provided the magnetic resonance imaging apparatus according to any one of (11) through (20), characterized in that the object is embedded in the object.

According to the aspect of the present invention, at least a unit of an embedded object is constituted by a substance having an atomic nucleus having a gyromagnetic ratio different from a gyromagnetic ratio of spin of an atomic nucleus of a substance occupying a majority in the object and accordingly, the object and the embedded object can be differentiated from each other by frequencies of the magnetic resonance signals. Thereby, the magnetic resonance imaging can be executed to differentiate the object from the embedded object.

Therefore, according to the present invention, there can be realized the intruded object capable of carrying out the magnetic resonance imaging in the state of being intruded into the object and the magnetic resonance imaging apparatus for capturing an image of the object having such an intruded object.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram of an apparatus of an example of embodiments of the present invention.

FIG. 2 is a diagram showing an example of pulse sequence executed by the apparatus shown in FIG. 1.

FIG. 3 is a diagram showing an example of pulse sequence executed by the apparatus shown in FIG. 1.

FIG. 4 is a conceptual view of piercing executed in parallel with image capturing.

FIG. 5 is a schematic constitution view of a piercing needle.

FIG. 6 is a conceptual diagram of Lamor frequency.

FIG. 7 is an apparatus block diagram shown in FIG. 1 from a view point of capturing an image accompanied by intervention.

FIG. 8 is a flowchart of operation of the apparatus shown in FIG. 1.

FIG. 9 is a schematic view of display image in the apparatus shown in FIG. 1.

FIG. 10 is a schematic view of display image in the apparatus shown in FIG. 1.

### DETAILED DESCRIPTION OF THE INVENTION

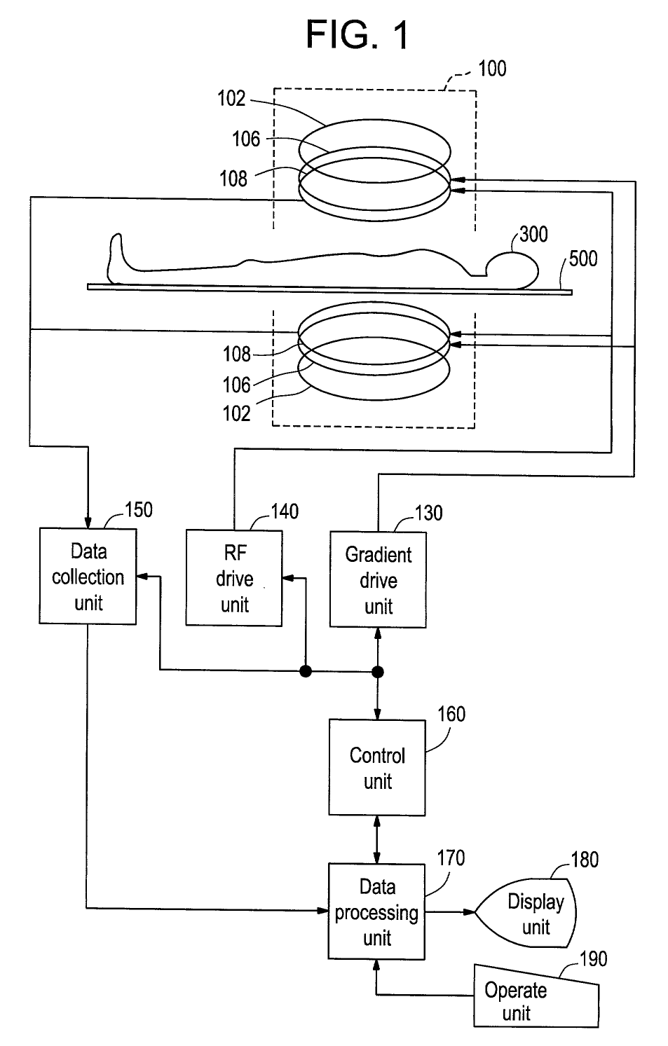

A detailed explanation will be given of embodiments of the present invention in reference to the drawings as follows. FIG. 1 shows a block diagram of a magnetic resonance imaging apparatus. The apparatus is an example of the embodiments of the present invention. By the constitution of the apparatus, there is shown an example of the embodiments with regard to the apparatus of the present invention.

As shown by FIG. 1, the apparatus is provided with a magnet system 100. The magnet system 100 is provided with main magnetic field magnet units 102, gradient coil units 106 and RF (radio frequency) coil units 108. The main magnetic field magnet units 102 and the respective coil units are respectively paired to be opposed to each other by interposing a space therebetween. Further, the respective units are provided with substantially a shape of a circular disk and are arranged by making central axes thereof common. An object 300 is mounted on a cradle 500 and carried in and carried out to and from an inner space (bore) of the magnet system 100 by carrying means, not illustrated.

The main magnetic field magnet units 102 form a magnetostatic field at the inner space of the magnet system 100. The direction of the magnetostatic field is substantially orthogonal to a direction of the body axis of the object 300. That is, so-to-speak vertical magnetic field is formed. The main magnetic field magnet units 102 are constituted by using, for example, a permanent magnet. Further, the main magnetic field magnet units 102 may naturally be constituted by using not only a permanent magnet but also a superconductive electromagnet or a normally conductive electromagnet.

The gradient coil units 106 generate gradient magnetic fields for providing gradients to an intensity of the magnetostatic field. The generated gradient magnetic fields are three kinds of a slice gradient magnetic field, a read out gradient magnetic field and a phase encode gradient magnetic field and in correspondence with the three kinds of gradient magnetic fields, the gradient coil units 106 are provided with three routes of gradient coils, not illustrated.

The RF coil units 108 form a high-frequency magnetic field for exciting spin in the body of the object 300 at the magnetostatic space. Hereinafter, formation of the high-frequency magnetic field is referred to as transmission of an RF excited signal. Further, the RF coil units 108 receive a magnetic resonance signal for generating excited spin. The RF coil units 108 are provided with a coil for transmission and a coil for reception, not illustrated. With regard to the coil for transmission and the coil for reception, the same coil is used therefor or exclusive coils are used respectively.

The gradient coil units 106 are connected with a gradient drive unit 130. The gradient drive unit 130 makes the gradient coil units 106 generate the gradient magnetic field by providing a drive signal thereto. The gradient drive unit 130 is provided with three routes of drive circuits, not illustrated, in correspondence with the three routes of gradient coils in the gradient coil units 106.

The RF coil units 108 are connected with an RF drive unit 140. The RF drive unit 140 transmits an RF excited signal by providing a drive signal to the RF coil units 108 to thereby excite spin in the body of the object 300.

Further, the RF coil unit 108 is connected with a data collecting unit 150. The data collecting unit 150 inputs a received signal received by the RF coil units 108 and collects the received signal as digital data.

The gradient drive unit 130, the RF drive unit 140 and the data collecting unit 150 are connected with a control unit 160. The control unit 160 executes to take an image by respectively controlling the gradient drive unit 130 through the data collecting unit 150.

An output side of the data correcting unit 150 is connected to a data processing unit 170. The data processing unit 170 is constituted by using, for example, a computer. The data processing unit 170 is provided with a memory, not illustrated. The memory is stored with programs and various data for the data processing unit 170. The function of the apparatus is realized by executing the programs stored to the memory by the data processing unit 170.

The data processing unit 170 stores data inputted from the data collecting unit 150 to the memory. A data space is formed in the memory. The data space constitutes a two-dimensional Fourier space. The data processing unit 170 forms (reconstructs) the image of the object 300 by subjecting data of the two-dimensional Fourier space to two-dimensional inverse Fourier transform. The two-dimensional Fourier space is referred to also as k-space.

The data processing unit 170 is connected to the control unit 160. The data processing unit 170 is higher than the control unit 160 and governs the control unit 160. The data processing unit 170 is further connected with a display unit 180 and an operating unit 190. The display unit 180 is constituted by a graphic display. The operating unit 190 is constituted by a keyboard having pointing devices such as a track ball or a mouse.

The display unit 180 displays a reconstructed image outputted from the data processing unit 170 and various information. The operating unit 190 is operated by an operator and inputs various instruction or information to the data processing unit 170. The operator interactively operates the apparatus via the display unit 180 and the operating unit 190.

A unit comprising the magnetic system 100 and the data collecting unit 150 is an example of embodiments of signal acquiring means according to the present invention. The data processing unit 170 is an example of a embodiments of image forming means according to the present invention. The display unit 180 is an example of embodiments of image displaying means according to the present invention. Further, the display unit 180 is an example of embodiments of position displaying means.

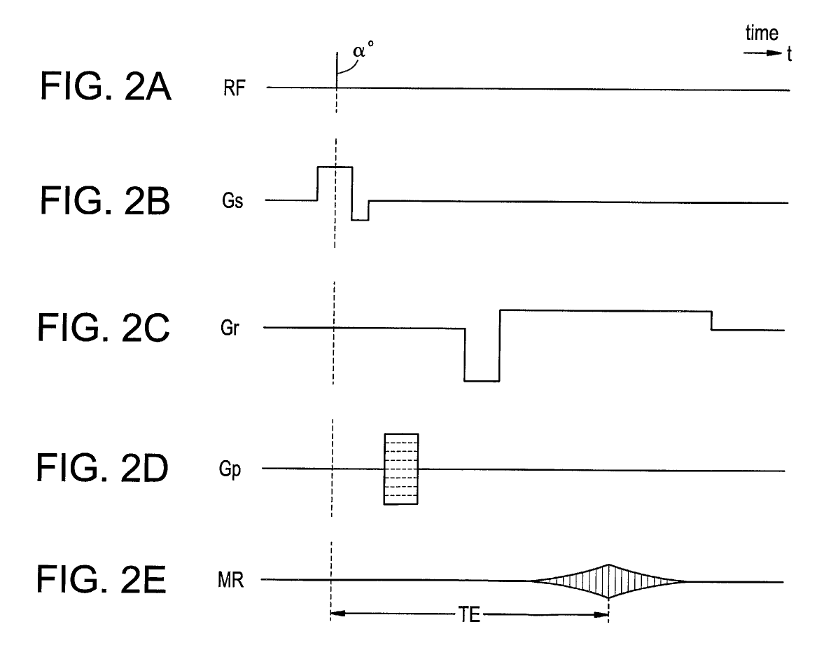

FIG. 2 shows an example of a pulse sequence used in magnetic resonance imaging. The pulse sequence is a pulse sequence of GRE (Gradient Echo) method.

That is, (1) of FIG. 2 indicates a sequence of a pulse for exciting RF in the GRE method and (2), (3), (4) and (5) thereof respectively indicate sequences of slice gradient Gs, read out gradient Gr, phase encode gradient Gp and gradient echo MR. Further, a pulse is represented by a central signal. The pulse sequence advances from left to right along a time axis t.

As shown by the drawing, α° excitation of spin is executed by α° pulse. The flip angle α° is equal to or smaller than 90°. At this occasion, the slice gradient Gs is applied and selective excitation is carried out with respect to predetermined slice.

After the α° excitation, phase encode of spin is executed by the phase encode gradient Gp. Next, spin is firstly dephased by the read out gradient Gr, successively, the spin is rephased and the gradient echo MR is generated. The signal intensity of the gradient echo MR is maximized at a time point after echo time TE from the α° excitation. The gradient echo MR is collected as view data by the data collecting unit 150.

The pulse sequence is repeated by 64 through 512 times by repetition time TR. At each repetition, the phase encode gradient Gp is changed and different phase encode is executed at each time. Thereby, there is provided view data of 64 through 512 views filling the k-space.

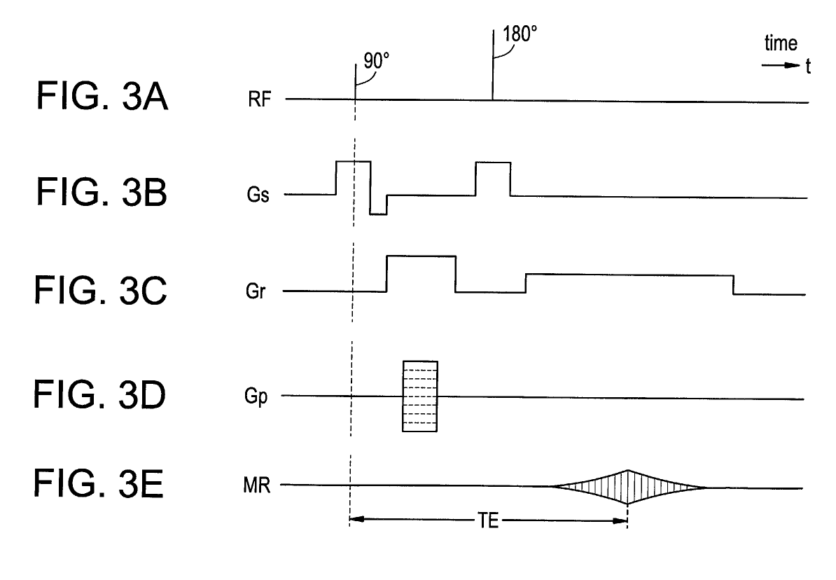

FIG. 3 shows other example of a pulse sequence for magnetic resonance imaging. The pulse sequence is a pulse sequence of SE (Spin Echo) method.

That is, (1) of FIG. 3 shows a sequence of 90° pulse and 180 pulse for exciting RF in the SE method and (2), (3), (4) and (5) thereof respectively indicate sequences of slice gradient Gs, read out gradient Gr, phase encode gradient Gp and spin echo MR. Further, the 90° pulse and the 180 pulse are respectively represented by central signals. The pulse sequence advances from left to right along the time axis t.

As shown by the drawing, 90° excitation of spin is executed by 90° pulse. At this occasion, the slice gradient Gs is applied and selective excitation is executed with respect to predetermined slice. After predetermined time from 90° excitation, 180 excitation by pulse, that is, spin inversion is executed. Also in this case, the slice gradient Gs is applied and selective inversion is executed with respect to the same slice.

During a time period between 90° excitation and spin inversion, there are applied the read out gradient Gr and the phase encode gradient Gp. Spin is dephased by the read out gradient Gr. Phase encode of spin is executed by the phase encode gradient Gp.

After the spin inversion, spin is rephased by the read out gradient Gr and spin echo MR is generated. The signal intensity of the spin echo MR is maximized at a time point after TE from 90° excitation. The spin echo MR is collected as view data by the data collecting unit 150.

Such a pulse sequence is repeated by 64 through 512 times by the repetition time TR. At each repetition, the phase encode gradient Gp is changed and different phase is executed at each time. Thereby, there are provided view data of 64 through 512 views filling the k-space.

Further, pulse sequence used in image capturing is not limited to the GRE method or the SE method but, for example, there may be used other pertinent method such as FSE (Fast Spin Echo) method, FSE (Fast Recovery Fast Spin Echo) method, and EPI (Echo Planar Imaging) and the like.

The data processing unit 170 reconstructs the laminar image of the object 300 by subjecting the view data of the k-space to two-dimensional inverse Fourier transform. The reconstructed image is stored to the memory or displayed at the display unit 180. In real time image capturing, such image capturing is executed continuously and reconstructed images are successively displayed.

For convenience of executing intervention such as piercing of the object 300 in parallel with real time image capturing of the laminar image, the display unit 180 may be arranged at a vicinity of the magnet system 100 to be capable of observing the laminar image at a side of the object 300. Or, a display unit prepared separately from the display unit 180 on the side of an operating chamber may be arranged at a vicinity of the magnet system 100.

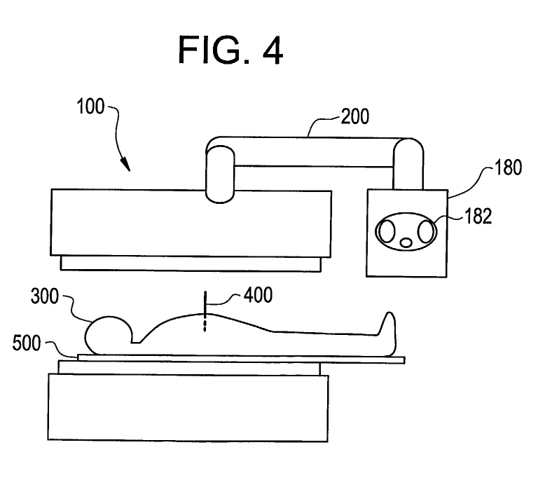

There is used such a display unit, for example, shown by FIG. 4. As shown by the drawing, the display unit 180 is supported by a support arm 200 extended from a cover. The support arm 200 is provided with pertinent joint units and a position of the display unit 180 and a direction of a display face thereof at a surrounding of the magnet system 100 are made adjustable. A laminar image 182 of the object 300 is displayed at the display unit 180. An operator, not illustrated, pierces the object 300 by a piercing needle 400 while observing the laminar image 182. The piercing needle 400 is an example of embodiments of an intruded object according to the present invention.

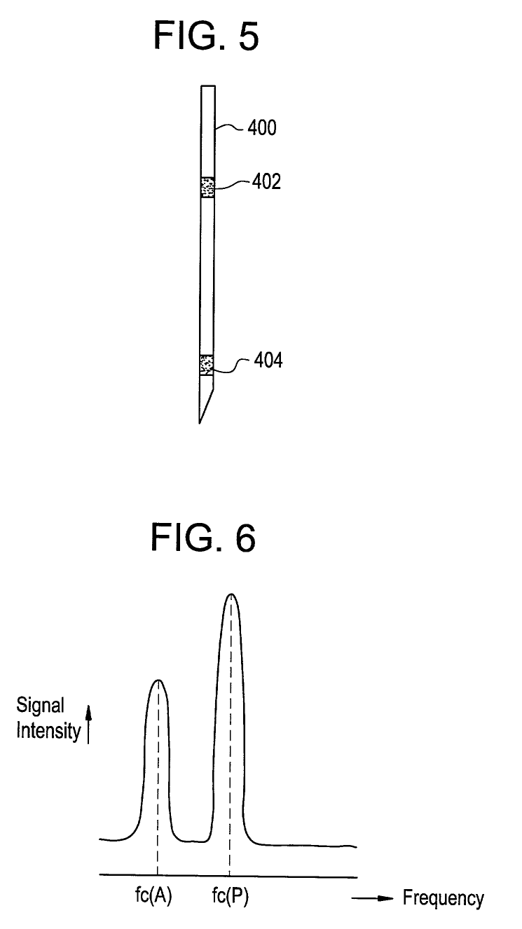

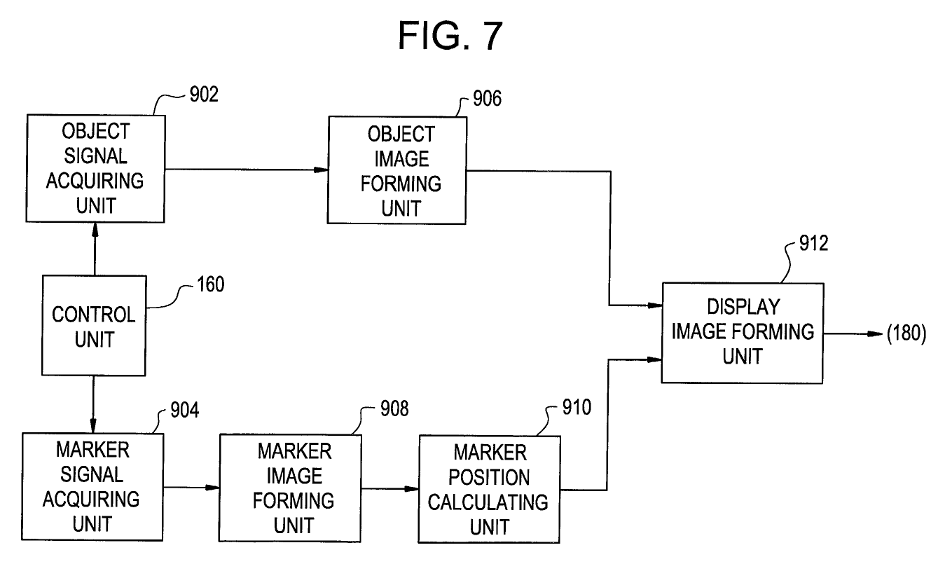

FIG. 5 shows a schematic constitution of the piercing needle 400. As shown by the drawing, the piercing needle 400 is provided with two markers 402 and 404. The markers 402 and 404 are provided with a predetermined interval therebetween in the length direction of the piercing needle 400. A number of the markers is not limited to two but may be one or may be three or more. Or, a total of the piercing needle 400 may be constituted by a substance the same as that of the markers 402 and 404.

As the substance constituting the markers 402 and 404, there is used a substance having an atomic nucleus having a gyromagnetic ratio different from a gyromagnetic ratio of spin of an atomic nucleus, that is, proton of a substance occupying a majority in the object 300. As such a substance, for example, silicon (Si) is used. Other than silicon, for example, N-acetylasparatate (NAA) is used. Either of these is preferable in view of being harmless to an organism. Silicon and NAA are provided with gyromagnetic ratios different from each other to a degree of being capable of differentiating each other.

The markers 402 and 404 may be constituted by the same substance or may be constituted by separate substances. When the markers are constituted by the same substance, it is preferable to make sizes of the markers differ from each other in order to facilitate to differentiate a front and a rear unit of the piercing needle 400.

By the difference in the gyromagnetic ratios, spin of the atomic nucleus of the markers 402 and 404 is provided with Lamor frequency fc (A) different from Lamor frequency fc (P) of spin of proton as is conceptually shown by FIG. 6. Thereby, the markers 402 and 404 can be provided with a magnetic resonance signal having a frequency different from that of the object 300 and based on the difference in the frequencies, images of the markers 402 and 404 can be taken to differentiate from the object 300.

It is preferable to take image by commonly using the same RF transmitting and receiving apparatus in view of simplifying the constitution. In contrast thereto, it is preferable to provide an RF retransmitting and receiving apparatus separately from that of an image capturing system exclusively for the markers in view of improving SNR (signal-to-noise ratio) of a received signal.

FIG. 6 shows a block diagram of the apparatus in view of capturing image accompanied by intervention. In the drawing, an object signal acquiring unit 902 acquires a magnetic resonance signal generated by proton in the object 300 and a marker signal acquiring unit 904 acquires magnetic resonance signals generated by the markers 402 and 404.

All of these correspond to units comprising the magnet system 100, the gradient drive unit 130, the RF drive unit 140 and the data collecting unit 150 in the apparatus shown by FIG. 1. The object signal acquiring unit 902 and the mark signal acquiring unit 904 are alternately operated under control by the control unit 160.

An object image forming unit 906 forms a laminar image of the object 300 based on the magnetic resonance signal acquired by the object signal acquiring unit 902. A marker image forming unit 908 forms images of the markers 402 and 404 based on the magnetic resonance signals acquired by the marker signal acquiring unit 904. Based on the images of the markers 402 and 404, a marker position calculating unit 910 calculates positions of the markers 402 and 404.

A display image forming unit 912 forms a display image based on the laminar image formed by the object image forming unit 906 and the positions of the markers calculated by the marker position calculating unit 910 and inputs the display image to the display unit 180. Units comprising the object image forming unit 906, the marker image forming unit 908, the marker position calculating unit 910 and the display image forming unit 912, correspond to the data processing unit 170 in the apparatus shown by FIG. 1.

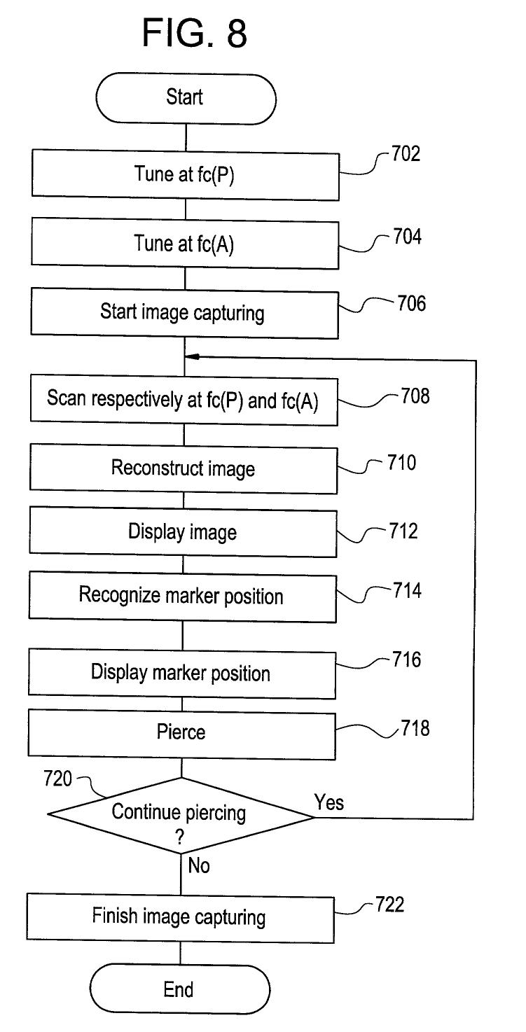

An explanation will be given of operation of the apparatus. FIG. 8 shows a flowchart of operation of the apparatus. As shown by the drawing, firstly, at step 702, tuning of the apparatus is carried out at frequency fc (P) and RF transmitting and receiving conditions more pertinently capturing an image of the object 300 are calculated.

Next, at step 704, tuning of the apparatus is carried out at frequency fc (A) and RF transmitting and receiving conditions for pertinently capturing images of the markers 402 and 404 are calculated. When frequencies of the markers 402 and 404 are different from each other, the tuning is carried out at respective frequencies.

Next, at step 706, image capturing is started. Thereby, at step 708, scanning at frequency fc (P) and scanning at fc (A) are respectively carried out. The scanning operations are carried out, for example, alternately at each scanning. Further, the operation is not limited thereto but scanning at fc (A) may be carried out once at respective plural times of scanning at frequency fc (P), or scanning at fc (A) may be carried out by plural times at each scanning at the frequency fc (P).

By the scanning at frequency fc (P), the magnetic resonance signal with respect to the object 300 is collected and by scanning at frequency fc (A), the magnetic resonance signals with respect to the markers 402 and 404 of the piercing needle 400 are collected. It is preferable to make steep the gradient of the gradient magnetic field in scanning at the frequency fc (A) in view of promoting spatial resolution of capturing the images of the markers 402 and 404.

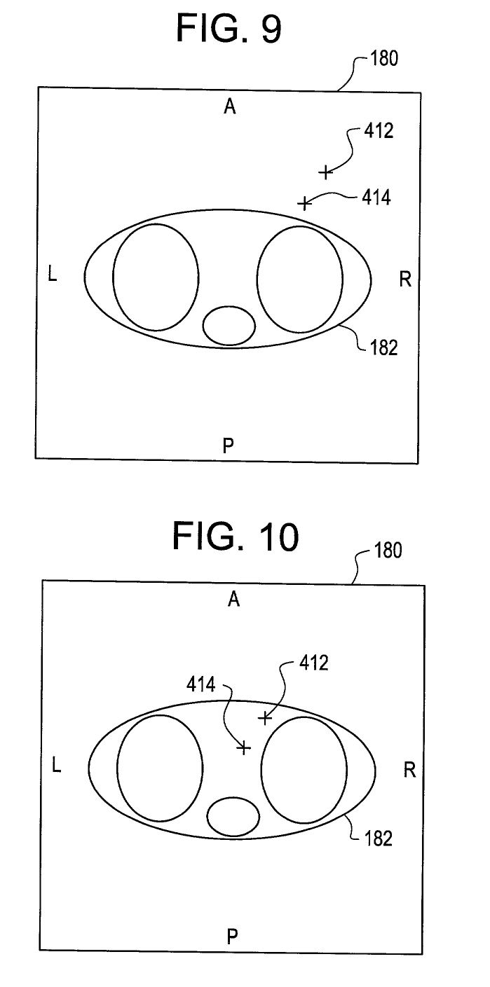

Next, at step 710, the laminar image of the object 300 is reconstructed. The laminar image of the object 300 is reconstructed based on the magnetic resonance signal collected by scanning at frequency fc (P). The reconstructed image is displayed at step 712. Thereby, for example, as shown by FIG. 9, the laminar image 182 of the object 300 is displayed at the display unit 180.

Next, at step 714, the positions of the markers are recognized. The positions of the markers are recognized by reconstructing the images of the markers 402 and 404 from the magnetic resonance signals collected by scanning at frequency fc (A) and calculating the positions of the images in the image capturing space. The recognized positions are displayed at step 716.

As shown by, for example, FIG. 9, the positions of the markers 402 and 404 are respectively indicated by predetermined marks 412 and 414 on the display screen of the laminar image 182. The shapes of the marks may be pertinent. The images of the markers 402 and 404 will do as they are. It is preferable that the marks 412 and 414 are displayed by color in view of improving visual recognition performance. When the frequencies of the markers 402 and 404 are different from each other, the display color may differ.

Based on the marks 412 and 414, the operator recognizes a positional relationship of the piercing needle 400 relative to an affected part to be pierced. By conceiving a straight line connecting the two marks 412 and 414, the inclination of the piercing needle 400 can easily be recognized. By forming and displaying the straight line connecting the marks 412 and 414, the position and the inclination of the piercing needle 400 can be grasped further easily.

In place of displaying in the above-described way or in addition thereto, the positions of the markers 402 and 404 may be displayed by numerically displaying the coordinates of the positions by the display unit 180 or a separate display.

Next, at step 718, the operator carries out the piercing operation. That is, the position and the direction of the piercing needle 400 are determined by confirming thereof at the display screen and the object 300 is pierced. In continuing to pierce the object 300, based on the determination at step 720, operation of steps 708 to 718 is repeated. Thereby, real time image capturing is carried out and piercing is carried out in parallel therewith.

In accordance with progress of the piercing needle 400, for example, as shown by FIG. 10, the marks 412 and 414 invade the inner unit of the laminar image 182. Thereby, the operator can accurately be informed of the position and the inclination of the piercing needle 400 at the inner unit of the object 300 and a desired part can accurately be pierced. The same is carried out also in the case of displaying the positions of the marks 412 and 414 by the coordinates.

When the scheduled piercing operation has been finished, the piercing needle 400 is drawn from the object 300 and at step 722, the image capturing is finished.

Although an explanation has been given of an example in which the intruded object is the piercing needle as described above, the intruded object is not limited to the piercing needle but may be an instrument inserted into the celom or an object embedded in the body and by attaching markers to such an instrument or object in accordance with the above-described, the position at the inner unit of the object can precisely be grasped.

Many widely different embodiments of the invention may be configured without departing from the spirit and the scope of the present invention. It should be understood that the present invention is not limited to the specific embodiments described in the specification, except as defined in the appended claims.

1. An intruded object which is a body intruded into an inner unit of an object for executing magnetic resonance imaging, wherein a unit of the body comprises a substance having an atomic nucleus having a gyromagnetic ratio different from a gyromagnetic ratio of spin of anatomic nucleus of a substance occupying a majority in the object.

2. The intruded object of claim 1, wherein the substance is harmless to an organism.

3. The intruded object of claim 2, wherein the substance is silicon.

4. The intruded object of claim 2, wherein the substance is N-acetylasparatate.

5. The intruded object of claim 1, wherein the substance comprises a plurality of substances having different gyromagnetic ratios of spin of atomic nuclei.

6. The intruded object of claim 5, wherein all of the plurality of substances are harmless to an organism.

7. The intruded object of claim 6, wherein one of the plurality of substances is silicon and other thereof is N-acetylasparatate.

8. The intruded object of claim 1, wherein the body pierces the object.

9. The intruded object of claim 1, wherein the body is inserted into a celom of the object.

10. The intruded object of claim 1, wherein the body is embedded in the object.

11. A magnetic resonance imaging apparatus comprising:
a signal acquiring device for acquiring magnetic resonance signals respectively of an object for executing magnetic resonance imaging and a body which is intruded into an inner unit of the object and at least a unit of which comprises a substance having an atomic nucleus having a gyromagnetic ratio different from a gyromagnetic ratio of spin of an atomic nucleus of a substance occupying a majority in the object; and
an image forming device for forming respectively an image of the object and an image of the intruded object based on the magnetic resonance signals.

12. The magnetic resonance imaging apparatus of claim 11, wherein the signal acquiring device alternately acquires the magnetic resonance signals with respect to the object and the body.

13. The magnetic resonance imaging apparatus of claim 11 further comprising:
an image displaying device for displaying the image of the object and the image of the intruded object on a same screen to differentiate from each other.

14. The magnetic resonance imaging apparatus of claim 11, further comprising:
a position displaying device for displaying a position of the intruded object based on the image of the intruded object.

15. The magnetic resonance imaging apparatus of claim 11, wherein the substance is harmless to an organism.

16. The magnetic resonance imaging apparatus according to claim 15, characterized in that the substance is silicon.

17. The magnetic resonance imaging apparatus of claim 15, wherein the substance is N-acetylasparatate.

18. The magnetic resonance imaging apparatus of claim 11, wherein the substance comprises a plurality of substances having different gyromagnetic ratios of spin of atomic nuclei.

19. The magnetic resonance imaging apparatus of claim 18, wherein all of the plurality of substances are harmless to an organism.

20. The magnetic resonance imaging apparatus of claim 19, wherein one of the plurality of substances is silicon and other thereof is N-acetylasparatate.

21. The magnetic resonance imaging apparatus of claim 11, wherein the body pierces the object.

22. The magnetic resonance imaging apparatus of claim 11, wherein the body is inserted into a celom of the object.

23. The magnetic resonance imaging apparatus of claim 11, wherein the object is embedded in the object.

24. A magnetic resonance imaging method comprising the steps of:
acquiring magnetic resonance signals respectively of an object for executing magnetic resonance imaging and a body which is intruded into an inner unit of the object and at least a unit of which comprises a substance having an atomic nucleus having a gyromagnetic ratio different from a gyromagnetic ratio of spin of an atomic nucleus of a substance occupying a majority in the object; and
forming respectively an image of the object and an image of the intruded object based on the magnetic resonance signals.
